(19) United States
(12) Patent Application Publication
(10) Pub. No.: US 2005/0182140 A1
(43) Pub. Date: Aug. 18, 2005

(54) Antimicrobial compositions

(75) Inventor: Stephen A. Payne, Charlotte, NC (US)

Correspondence Address: KENNEDY COVINGTON LOBDELL & HICKMAN, LLP, 214 N. TRYON STREET, HEARST TOWER, 47TH FLOOR, CHARLOTTE, NC, 28202, US

(73) Assignee: Microban Products Company, Huntersville, NC (US)

(21) Appl. No.: 10/999,549

(22) Filed: Nov. 30, 2004

Related U.S. Application Data
(60) Provisional application No. 60/525,910, filed on Dec. 1, 2003.
(60) Provisional application No. 60/551,485, filed on Mar. 9, 2004.

(51) Int. Cl. A61K 31/14; A61K 31/05

(52) U.S. Cl. 514/643; 514/731

(57) ABSTRACT

An antimicrobial composition imparts antimicrobial characteristics to numerous products. The antimicrobial composition comprises an emulsion, wherein the emulsion comprises a quaternary ammonium antimicrobial agent, an alkyl phenol, a styrenated phenol and water. A method for producing this composition is also disclosed.

### CROSS-REFERENCE TO RELATED APPLICATIONS

The present application claims priority from U.S. provisional application No. 60/525,910, filed on Dec. 1, 2003, and U.S. provisional application No. 60/551,485, filed on Mar. 9, 2004, each of which is incorporated herein by reference.

### FIELD OF THE INVENTION

The invention relates to the field of antimicrobial protection. More specifically, the invention relates to a composition for imparting built-in and long lasting antimicrobial characteristics to various products. In particular, the invention pertains to a novel aqueous emulsion of a quaternary ammonium antimicrobial agent.

### BACKGROUND OF THE INVENTION

The field of providing products with built-in antimicrobial protection has grown tremendously over the past several years. What once was a premium or novel option for high-end consumer products and medical devices has grown into a mainstream characteristic found in many consumer products. Consumers can go to any home improvement center and see dozens if not hundreds of products that claim some degree of resistance to microbiological growth or contamination. Some major retailers have specific sections devoted to such antimicrobial products. Microban Products Company, the assignee of the present application, has several patents that are representative of the work and research that is currently ongoing in this area.

One of the challenges faced in all built-in antimicrobial applications is matching an effective antimicrobial agent with a particular product. For example, one antimicrobial agent may work well in interior applications (e.g., interior paint) yet be unsuitable for some outdoor applications (e.g., house siding). Similarly, an agent that works well against one type of microbe (e.g., fungi) may not work against another type of microbe (e.g., bacteria).

Accordingly, imparting antimicrobial characteristics to particular products is not simply a matter of pulling an antimicrobial agent off of a shelf and adding it to an existing product. Many variables must be considered and sometimes a commercially acceptable solution (i.e., effective and economically acceptable) cannot be found. Furthermore, as the field of built-in antimicrobial protection grows, each new product presents researchers with a new set of problems.

Therefore, a continuing need exists for new antimicrobial compositions that can be added to the arsenal of weapons used to fight the proliferation of microbes on and in consumer and industrial products.

### SUMMARY OF THE INVENTION

The present invention provides a new and useful antimicrobial composition that can impart antimicrobial characteristics in a wide range of products.

The present invention also provides a method of manufacturing a new and useful antimicrobial composition.

In one embodiment, an antimicrobial composition comprises an emulsion where the emulsion comprises a quaternary ammonium antimicrobial agent, an alkyl phenol, a styrenated phenol, and water.

In another embodiment, a process for making an antimicrobial composition comprises blending an alkyl phenol with a quaternary ammonium antimicrobial agent, heating the blended alkyl phenol and the quaternary ammonium antimicrobial agent, admixing a quantity of styrenated phenol, and admixing a quantity of water.

### DETAILED DESCRIPTION OF THE INVENTION

As used herein, the terms “microbe” or “microbial” should be interpreted to encompass any of the microscopic organisms commonly studied by microbiologists. Such organisms include, but are not limited to, bacteria and fungi as well as other single-celled organisms such as mold, mildew and algae. Viral particles and other infectious agents are also included in the term microbe.

The term “antimicrobial” includes biostatic activity, i.e., where the proliferation of microbiological species is reduced or eliminated, and true biocidal activity where microbiological species are killed. For ease of discussion, this detailed description may make reference to bacteria and antibacterial agents. This method of presentation should not be interpreted as limiting the scope of the invention in any way.

The term efficacy, as used herein, is defined as the characteristic of inhibiting the growth of a microbe on a substrate.

In broad terms, the invention is an antimicrobial composition comprising an emulsion of a quaternary ammonium antimicrobial agent, two different types of phenols, and water.

Turning now to more specific embodiments of the invention, one embodiment of the invention is an antimicrobial composition that can impart antimicrobial characteristics to many different products. In its most basic form, this embodiment of the invention comprises an aqueous emulsion. The emulsion comprises a quaternary ammonium antimicrobial agent, an alkyl phenol, a styrenated phenol, and water. Interestingly, the composition need not contain the volatile alcohols (e.g., ethanol) that are usually used to form emulsions of quaternary ammonium antimicrobial agents. Each of these elements, and other preferred and optional elements, will be discussed in more detail below.

Quaternary ammonium antimicrobial agents include, but are not limited to, N-alkyldimethyl benzyl ammonium saccharinate, 1,3,5-Triazine-1,3,5(2H,4H,6H)-triethanol; 1-Decanaminium, N-decyl-N,N-dimethyl-, chloride (or) Didecyl dimethyl ammonium chloride; 2-(2-(p-(Diisobuyl)cresosxy)ethoxy)ehyl dimethyl benzyl ammonium chloride; 2-(2-(p-(Diisobutyl)phenoxy)ethoxy)ethyl dimethyl benzyl ammonium chloride; alkyl 1 or 3 benzyl-1-(2-hydroxethyl)-2-imidazolinium chloride; alkyl bis(2-hydroxyethyl)benzyl ammonium chloride; alkyl demethyl benzyl ammonium chloride; alkyl dimethyl 3,4-dichlorobenzyl ammonium chloride (100% C12); alkyl dimethyl 3,4-dichlorobenzyl ammonium chloride (50% C14, 40% C12, 10% C16); alkyl dimethyl 3,4-dichlorobenzyl ammonium chloride (55% C14, 23% C12, 20% C16); alkyl dimethyl benzyl ammonium chloride; alkyl dimethyl benzyl ammonium chloride (100% C14); alkyl dimethyl benzyl ammonium chloride (100% C16); alkyl dimethyl benzyl ammonium chloride (41% C14, 28% C12); alkyl dimethyl benzyl ammonium chloride (47% C12, 18% C14); alkyl dimethyl benzyl ammonium chloride (55% C16, 20% C14); alkyl dimethyl benzyl ammonium chloride (58% C14, 28% C16); alkyl dimethyl benzyl ammonium chloride (60% C14, 25% C12); alkyl dimethyl benzyl-ammonium chloride (61% C11, 23% C14); alkyl dimethyl benzyl ammonium chloride (61% C12, 23% C14); alkyl dimethyl benzyl ammonium chloride (65% C12, 25% C14); alkyl dimethyl benzyl ammonium chloride (67% C12, 24% C14); alkyl dimethyl benzyl ammonium chloride (67% C12, 25% C14); alkyl dimethyl benzyl ammonium chloride (90% C14, 5% C12); alkyl dimethyl benzyl ammonium chloride (93% C14, 4% C12); alkyl dimethyl benzyl ammonium chloride (95% C16, 5% C18); alkyl dimethyl benzyl ammonium chloride (and) didecyl dimethyl ammonium chloride; alkyl dimethyl benzyl ammonium chloride (as in fatty acids); alkyl dimethyl benzyl ammonium chloride (C12-C16); alkyl dimethyl benzyl ammonium chloride (C12-C18); alkyl dimethyl benzyl and dialkyl dimethyl ammonium chloride; alkyl dimethyl dimethy benzyl ammonium chloride; alkyl dimethyl ethyl ammonium bromide (90% C14, 5% C16, 5% C12); alkyl dimethyl ethyl ammonium bromide (mixed alkyl and alkenyl groups as in the fatty acids of soybean oil); alkyl dimethyl ethylbenzyl ammonium chloride; alkyl dimethyl ethylbenzyl ammonium chloride (60% C14); alkyl dimethyl isoproylbenzyl ammonium chloride (50% C12, 30% C14, 17% C16, 3% C18); alkyl trimethyl ammonium chloride (58% C18, 40% C16, 1% C14, 1% C12); alkyl trimethyl ammonium chloride (90% C18, 10% C16); alkyldimethyl(ethylbenzyl)ammonium chloride (C12-18); Di-(C8-10)-alkyl dimethyl ammonium chlorides; dialkyl dimethyl ammonium chloride; dialkyl dimethyl ammonium chloride; dialkyl dimethyl ammonium chloride; dialkyl methyl benzyl ammonium chloride; didecyl dimethyl ammonium chloride; diisodecyl dimethyl ammonium chloride; dioctyl dimethyl ammonium chloride; dodecyl bis(2-hydroxyethyl)octyl hydrogen ammonium chloride; dodecyl dimethyl benzyl ammonium chloride; dodecylcarbamoyl methyl dimethyl benzyl ammonium chloride; heptadecyl hydroxyethylimidazolinium chloride; hexahydro-1,3,5-thris(2-hydroxyethyl)-s-triazine; myristalkonium chloride (and) Quat RNIUM 14; N,N-Dimethyl-2-hydroxypropylammonium chloride polymer; n-alkyl dimethyl benzyl ammonium chloride; n-alkyl dimethyl ethylbenzyl ammonium chloride; n-tetradecyl dimethyl benzyl ammonium chloride monohydrate; octyl decyl dimethyl ammonium chloride; octyl dodecyl dimethyl ammonium chloride; octyphenoxyethoxyethyl dimethyl benzyl ammonium chloride; oxydiethylenebis(alkyl dimethyl ammonium chloride); quaternary ammonium compounds, dicoco alkyldimethyl, chloride; trimethoxysily propyl dimethyl octadecyl ammonium chloride; trimethoxysilyl quats, trimethyl dodecylbenzyl ammonium chloride; n-dodecyl dimethyl ethylbenzyl ammonium chloride; n-hexadecyl dimethyl benzyl ammonium chloride; n-tetradecyl dimethyl benzyl ammonium chloride; n-tetradecyl dimethyl ethyylbenzyl ammonium chloride; and n-octadecyl dimethyl benzyl ammonium chloride.

Preferably, the antimicrobial agent is a water insoluble quaternary ammonium antimicrobial agent. More preferably, the antimicrobial agent is a non-halogenated benzyl substituted quaternary ammonium antimicrobial agent having the following structure: 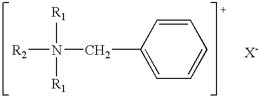
where
   R₁ is methyl or hydroxyethyl;
   R₂ is CₙH₂ₙ+1;
   n is 8 to 18, preferably 12, 14, or 16; and
   X is an anionic species.

Such agents include, but are not limited to, N-alkyldimethyl benzyl ammonium saccharinate; alkyl dimethyl ethylbenzyl ammonium cyclohexylsulfamate; alkyl bis(2-hydroxyethyl)benzyl ammonium halide; alkyl dimethyl 1-napthylmethyl ammonium halide; alkyl dodecylbenzyl dimethyl ammonium halide; and alkylbenzyl trimethyl ammonium halide.

In particularly preferred embodiments the quaternary ammonium antimicrobial agent comprises a dimethylbenzyl ammonium compound such as N-alkyl dimethylbenzyl ammonium saccharinate. N-alkyl dimethylbenzyl ammonium saccharinate is commercially available from Stepan Chemical Company of Northfield, Ill., under the tradename ONYXIDE™ 3300. This particular form of ONYXIDE™ is approximately 95% active and is a solid at room temperature but will form a liquid at elevated temperature. It is light yellow-orange in color and is insoluble in water.

The alkyl phenol utilized in the practice of the invention preferably comprises at least one alkyl phenol having at least one alkyl group-selected from the group consisting of C₇ alkyls, C₈ alkyls, C₉ alkyls, C₁₀ alkyls, and C₁₁ alkyls.

In most preferred embodiments the alkyl phenol comprises an alkyl phenol having a C₉ alkyl group.

Alkyl phenols suitable for use in the practice of the present invention are available commercially from a number of sources. A particularly preferred commercially available alkyl phenol is sold by Dow Chemical Company under the tradename TRITON™ X-207.

The styrenated phenol utilized in the practice of the invention is preferably non-ionic. In preferred embodiments the styrenated phenol is CHROMASIST™ WEZ which is commercially available from Cognis Corporation whose North American office is in Cincinnati, Ohio.

Water makes up the other primary component of the claimed antimicrobial composition.

The antimicrobial composition according to the invention may contain other additives. Two such additives are anti-foaming agents and anti-freezing agents.

The alkyl phenols and styrenated phenols used in the practice of the invention can be susceptible to foaming depending upon the particular application. Therefore, it is envisioned that some commercial embodiments of the invention will contain anti-foaming agents.

For example, the composition according to the invention may be used to treat kraft paper. Such paper is used to form the outer surface on gypsum wallboard. Kraft paper is also used as backing for insulation material that is often used in residential and commercial construction. One method of treating this type of paper is to add the antimicrobial composition to the water box, a device toward the end of the paper process that returns a certain quantity of water to the paper after the paper has undergone heat based drying.

The water box is often agitated due to the continuous movement of paper through it. This agitation can cause foaming upon the addition of the claimed antimicrobial composition. Generally, the presence of foam is detrimental to the water box portion of a paper process. Therefore an anti-foaming agent is added to the composition according to the invention when the invention is used to treat paper at the water box.

Preferred anti-foaming agents are ethoxylated co-polymers of polyethylene glycol. In some instances, the commerical formulations of stryrenated phenols and alkyl phenols may contain a quantity of an antifoaming agent. For example, TRITON™ X-207 contains a small quantity of polyethylene glycol. Accordingly, if TRITON™ X-207 is the source of the alkyl phenol additional antifoaming agent may not be needed.

Likewise, anti-freezing agents may be added to the composition according to the invention. The anti-freezing agents are not thought to be critical to the actual performance of the invention. Instead they are used to keep the composition from freezing or becoming too viscous during transport in cold weather. The anti-freezing agents may be left out of the composition if they are found to interfere with the addition of the antimicrobial agent in any particular process. A preferred anti-freezing agent is dipropylene glycol.

The relative quantities of each of the listed components may vary to accommodate particular process requirements. The versatility of the invention is discussed in more detail below but generally it should be recognized that the optimal formulation for one product (e.g., a polymer) may be different from the optimal formulation for another product (e.g., paper). Accordingly, each of the listed components may be present in different amounts depending upon the particular needs of the user. Again, those skilled in the art are fully capable of making these adjustments without undue experimentation.

In preferred embodiments the quaternary ammonium antimicrobial agent is present in the overall composition in an amount from about 0 wt. % to about 30 wt. %. In particularly preferred embodiments the quaternary ammonium antimicrobial agent is present from about 15 wt. % to about 20 wt. % of the overall composition.

As an example of how the invention may be tailored to meet specific process requirements, it was determined that a composition having approximately 17 wt. % ONYXIDE™ 3300 successfully and efficiently imparted antimicrobial characteristics to wallboard paper in one particular papermaking process.

Likewise, the alkyl phenol is preferably present in the overall composition in an amount from about 20 wt. % to about 50 wt. % of the total composition. In preferred embodiments the alkyl phenol is present in the composition from about 35 wt. % to about 45 wt. %. Continuing with the wallboard example, trial work on the same paper process determined that a concentration of about 44 wt. % alkyl phenol (TRITON™ X-207) was optimum for that particular process.

In preferred embodiments the styrenated phenol is present in the composition from about 1 wt. % to about 10 wt. %. Continuing with the wallboard example, trial work on the same paper process determined that a concentration of about 5 wt. % styrenated phenol (CHROMASIST™ WEZ) was optimum for that particular process.

It is anticipated that in most applications the quantity of anti-foaming agent needed for successful practice of the invention will range from about 0 wt. % to about 20 wt. %, more preferably from about 10 wt. % to about 16 wt. %. In the wallboard trials the ethoxylated co-polymer used as the anti-foaming agent was the polyethylene glycol that was already present in the TRITON™ X-207.

The remainder of the composition according to the invention comprises water. In a preferred embodiment, the quantity of water present is from about 7 wt. % to about 20 wt. %, more preferably from about 10 wt. % to about 17 wt. %. Again, the exact quantity of water will depend upon the particular application and those skilled in the art are capable of making the necessary adjustments.

Further embodiments of the invention include those products that incorporate the claimed antimicrobial composition. Indeed, one of the novel aspects of the invention is that it serves as a very versatile tool for incorporating quaternary ammonium antimicrobial agents into a variety of products. For example, the antimicrobial composition according to the invention has been shown to be particularly effective at imparting antimicrobial characteristics to paper used in the production of wallboard.

Preliminary work also indicates that the antimicrobial composition according to the invention is an excellent tool for providing antimicrobial protection to products such as paint and polymers, with latex exterior paints and extruded vinyl (e.g., vinyl siding, vinyl windows) being particularly suitable for use with the invention. It is also anticipated that the antimicrobial composition according to the invention can be added to other solids such as ceramics and cementitious binders to impart antimicrobial characteristics.

The invention also encompasses a method of making an antimicrobial composition. In broad terms, the method according to the invention comprises the steps of blending an alkyl phenol with a quaternary ammonium antimicrobial agent, heating the blended alkyl phenol and quaternary ammonium antimicrobial agent, admixing a quantity of a styrenated phenol, and admixing a quantity of water.

In preferred embodiments, the alkyl phenol (e.g., TRITON™ X-207) is blended with the quaternary ammonium antimicrobial agent (e.g., ONYXIDE™) in the presence of heat. The heat is applied because in many instances the quaternary ammonium antimicrobial agent is a solid at room temperature. Care should be taken not to heat the admixture of alkyl phenol and antimicrobial agent to a point where there is unacceptable volatilization of either.

If TRITON™ X-207 is the alkyl phenol and ONYXIDE™ is the antimicrobial agent, a mixing temperature of from about 65° C. to about 75° C. is recommended. At this temperature the ONYXIDE™ melts into the TRITON™ X-207 to form a liquid.

The styrenated phenol (e.g., CHROMASIST™ WEZ) is then admixed to the alkyl phenol and antimicrobial agent mixture. Heating can continue during this step if needed. Anti-foaming agents and anti-freezing agents such as those discussed previously can be added at this point if needed or desired.

Once the antimicrobial agent, alkyl phenol, and styrenated phenol are mixed heat may be removed. As the admixture cools to room temperature water is added with stirring.

The relative amounts of antimicrobial agent, alkyl phenol, styrenated phenol, and water utilized in the practice of the method according to the invention are the same as those discussed in relation to the composition according to the invention. Likewise, the relative amounts of anti-foaming agents, anti-freezing agents, and additional antimicrobial agents, if used are the same as those discussed in relation to the composition according to the invention.

### EXAMPLES

### Example 1

A one pound emulsion with water and dipropylene glycol was prepared and is referred to as Emulsion A. The emulsion comprised the following:

Emulsion A

| Component | Weight Percentage (%) |
|---|---|
| ONYXIDE ™ 3300 | 19 |
| TRITON ™ X-207 | 44 |
| MDS-42 | 15 |
| CHROMASIST ™ WEZ | 5 |
| dipropylene glycol | 5 |
| water | 12 |

In order to prepare Emulsion A, the TRITON™ X-207 was heated to 85° C. The ONYXIDE™ 3300 was pre-melted and added to the X-207 with stirring. The MDS-42, CHROMASIST™ WEZ, and dipropylene glycol were then added with heat and stirring. The mixture was allowed to cool slightly before adding the water (50 to 56° C.) to minimize evaporation. It was mixed at a medium-slow speed until a clear homogeneous solution was produced. The emulsion had a clear yellow tint and a slight odor. The ONYXIDE™ 3300 was 95% active. The viscosity at 25° C. was 732. The specific gravity was 1. The pH was 5-6.

### Example 2

The efficacy of Emulsion A in cotton sample material was tested, alone and in combination with other agents. The AATCC Test Method 147, which is a known bacterial test method for textiles, was followed. The sample size of the cotton was a 25 mm×75 mm rectangle. The growth medium was Nutrient Agar. Prior to treatment, the cotton was steam sterilized at 121° C. The samples were incubated at 37° C.±2° C. for 18 to 24 hours. The samples were each laundered 50 times prior to doing the micro testing unless indicated to the contrary. NZ means that no zone of inhibition surrounded the sample. NI means that there was no inhibition of growth under the sample (if observable). The zone of inhibition was reported in millimeters.

| | | | | Size |
|---|---|---|---|---|
| | | | | of |
| Sample | | | Zone | Zone |
| No. | Sample Description | Organism | Results | (mm) |
| 1 | Emulsion A at 2000 ppm | K. pneumoniae | NZ/NI | 0 |
| 2 | Emulsion A at 2000 ppm | S. aureus 6538 | I | 3 |
| 3 | COSMOCIL ™ CQ (Arch | K. pneumoniae | NZ/NI | 0 |
| | Chemical, Inc.) at 1200 ppm | | | |
| | and Emulsion A at | | | |
| | 1800 ppm | | | |
| 4 | CQ at 1200 ppm and | S. aureus 6538 | I | 9 |
| | Emulsion A at 1800 ppm | | | |
| 5 | CQ at 400 ppm, Emulsion | K. pneumoniae | NZ/NI | 0 |
| | A at 400 ppm and FPS at | | | |
| | 400 ppm | | | |
| 6 | CQ at 400 ppm, Emulsion | S. aureus 6538 | I | 6 |
| | A at 400 ppm and FPS at | | | |
| | 400 ppm | | | |
| 7 | CQ at 400 ppm and FPS | K. pneumoniae | I | 3 |
| | at 600 ppm | | | |
| 8 | CQ at 400 ppm and FPS | S. aureus 6538 | I | 5 |
| | at 600 ppm | | | |
| 9 | Zinc pyrithione (FPS | K. pneumoniae | NZ/NI | 0 |
| | dispersion) (Arch | | | |
| | Chemical, Inc.) at 900 ppm | | | |
| 10 | Zinc pyrithione (FPS | S. aureus 6538 | I | 2 |
| | dispersion) at 900 ppm | | | |
| 11 | Sodium OMADINE ™ | K. pneumoniae | NZ/NI | 0 |
| | 40% Aqueous Solution | | | |
| | (Arch Chemical, Inc.) at | | | |
| | 900 ppm | | | |
| 12 | Sodium OMADINE ™ | S. aureus 6538 | NZ/I | 0 |
| | 40% Aqueous Solution at | | | |
| | 900 ppm | | | |
| 13 | CQ at 400 ppm and | K. pneumoniae | NZ/I | 0 |
| | Sodium OMADINE ™ | | | |
| | 40% Aqueous Solution at | | | |
| | 600 ppm | | | |
| 14 | CQ at 400 ppm and | S. aureus 6538 | I | 1 |
| | Sodium OMADINE ™ | | | |
| | 40% Aqueous Solution at | | | |
| | 600 ppm | | | |
| 15 | CQ at 400 ppm, Emulsion | K. pneumoniae | NZ/NI | 0 |
| | A at 400 ppm and Sodium | | | |
| | OMADINE ™ 40% | | | |
| | Aqueous Solution at 400 ppm | | | |
| 16 | CQ at 400 ppm, Emulsion | S. aureus 6538 | I | 4 |
| | A at 400 ppm and Sodium | | | |
| | OMADINE ™ 40% | | | |
| | Aqueous Solution at 400 ppm | | | |
| 17 | CQ at 200 ppm and | K. pneumoniae | NZ/NI | 0 |
| | Sodium OMADINE ™ | | | |
| | 40% Aqueous Solution at | | | |
| | 400 ppm | | | |
| 18 | CQ at 200 ppm and | S. aureus 6538 | NZ/NI | 0 |
| | Sodium OMADINE ™ | | | |
| | 40% Aqueous Solution at | | | |
| | 400 ppm | | | |
| 19 | CQ at 200 ppm, Emulsion | K. pneumoniae | NZ/NI | 0 |
| | A at 200 ppm and Sodium | | | |
| | OMADINE ™ 40% | | | |
| | Aqueous Solution at 200 ppm | | | |
| 20 | CQ at 200 ppm, Emulsion | S. aureus 6538 | I | 3 |
| | A at 200 ppm and Sodium | | | |
| | OMADINE ™ 40% | | | |
| | Aqueous Solution at 200 ppm | | | |
| 21 | CQ at 900 ppm | K. pneumoniae | NZ/NI | 0 |
| 22 | CQ at 900 ppm | S. aureus 6538 | NZ/NI | 0 |
| 23 | Control | K. pneumoniae | NZ/NI | 0 |
| 24 | Control | S. aureus 6538 | NZ/NI | 0 |
| 25 | Control - no launderings | K. pneumoniae | NZ/NI | 0 |
| 26 | Control - no launderings | S. aureus 6538 | NZ/NI | 0 |

### Example 3

The efficacy of Emulsion A in cotton sample material was tested, alone and in combination with other agents. The Kirby Bauer Test Method, which is a known bacterial test method, was followed. The sample size of the cotton was 38.1 mm×38.1 mm (1.5 inches×1.5 inches) square. The growth medium was Mueller-Hinton Agar. Prior to treatment, the cotton was steam sterilized at 121° C. The samples were pre-wet with sterile deionized water. The samples were incubated at 37° C.±2° C. for 18 to 24 hours. The samples were each laundered 50 times unless indicated to the contrary. NZ means that no zone of inhibition surrounded the sample. NI means that there was no inhibition of growth under the sample (if observable). The zone of inhibition was reported in millimeters.

| | | | | Size |
|---|---|---|---|---|
| | | | | of |
| Sample | | | Zone | Zone |
| No. | Sample Description | Organism | Results | (mm) |
| 1 | Emulsion A at 800 ppm, | K. pneumoniae | NZ/I | 0 |
| | R10800 (Arkophob | | | |
| | DAN) at 0.67%, and | | | |
| | USV (Bayer) at 0.33% | | | |
| 2 | Emulsion A at 800 ppm, | S. aureus 6538 | NZ/I | 0 |
| | R10800 at 0.67%, and | | | |
| | USV at 0.33% | | | |
| 3 | Triclocarban (Bayer) at | K. pneumoniae | NZ/NI | 0 |
| | 800 ppm, R10800 at | | | |
| | 0.67%, and USV at | | | |
| | 0.33% | | | |
| 4 | Triclocarban at 800 ppm, | S. aureus 6538 | NZ/NI | 0 |
| | R10800 at 0.67%, | | | |
| | and USV at 0.33% | | | |
| 5 | BETHOGUARD | K. pneumoniae | NZ/NI | 0 |
| | (Janssen) at 800 ppm, | | | |
| | R10800 at 0.67%, and | | | |
| | USV at 0.33% | | | |
| 6 | BETHOGUARD at 800 ppm, | S. aureus 6538 | NZ/NI | 0 |
| | R10800 at 0.67%, | | | |
| | and USV at 0.33% | | | |
| 7 | BUBOND 60 (Buckman | K. pneumoniae | NZ/NI | 0 |
| | Chemical) at 800 ppm, | | | |
| | R10800 at 0.67%, and | | | |
| | USV at 0.33% | | | |
| 8 | BUBOND 60 at 800 ppm, | S. aureus 6538 | NZ/NI | 0 |
| | R10800 at 0.67%, | | | |
| | and USV at 0.33% | | | |
| 9 | Zinc pyrithione (FPS | K. pneumoniae | NZ/NI | 0 |
| | dispersion) at 800 ppm, | | | |
| | R10800 at 0.67%, and | | | |
| | USV at 0.33% | | | |
| 10 | Zinc pyrithione (FPS | S. aureus 6538 | NZ/I | 0 |
| | dispersion) at 800 ppm, | | | |
| | R10800 at 0.67%, and | | | |
| | USV at 0.33% | | | |
| 11 | Ortho-phenyl phenol at | K. pneumoniae | NZ/NI | 0 |
| | 800 ppm, R10800 at | | | |
| | 0.67%, and USV at | | | |
| | 0.33% | | | |
| 12 | Ortho-phenyl phenol at | S. aureus 6538 | NZ/NI | 0 |
| | 800, R10800 ppm at | | | |
| | 0.67%, and USV at | | | |
| | 0.33% | | | |
| 13 | Polyhexamethylene | K. pneumoniae | NZ/NI | 0 |
| | biguanide (PHMB) at | | | |
| | 534 ppm, R10800 at | | | |
| | 0.67%, and USV at | | | |
| | 0.33% | | | |
| 14 | PHMB at 534 ppm, | S. aureus 6538 | NZ/NI | 0 |
| | R10800 at 0.67%, and | | | |
| | USV at 0.33% | | | |
| 15 | PHMB at 356 ppm, FPS | K. pneumoniae | NZ/NI | 0 |
| | at 533 ppm, R10800 at | | | |
| | 0.67%, and USV at | | | |
| | 0.33% | | | |
| 16 | PHMB at 356 ppm, FPS | S. aureus 6538 | NZ/I | 0 |
| | at 533 ppm, R10800 at | | | |
| | 0.67%, and USV at | | | |
| | 0.33% | | | |
| 17 | PHMB at 356 ppm | K. pneumoniae | NZ/NI | 0 |
| | Emulsion A at 533 ppm, | | | |
| | R10800 at 0.67%, and | | | |
| | USV at 0.33% | | | |
| 18 | PHMB at 356 ppm, | S. aureus 6538 | NZ/NI | 0 |
| | Emulsion A at 533 ppm, | | | |
| | R10800 at 0.67%, and | | | |
| | USV at 0.33% | | | |
| 19 | BUBOND 60 at 356 ppm, | K. pneumoniae | NZ/I | 0 |
| | FPS at 533 ppm, | | | |
| | R10800 at 0.67%, and | | | |
| | USV at 0.33% | | | |
| 20 | BUBOND 60 at 356 ppm, | S. aureus 6538 | NZ/I | 0 |
| | FPS at 533 ppm, | | | |
| | R10800 at 0.67%, and | | | |
| | USV at 0.33% | | | |
| 21 | BUBOND 60 at 356 ppm, | K. pneumoniae | NZ/NI | 0 |
| | Emulsion A at 533 ppm, | | | |
| | R10800 at 0.67%, | | | |
| | and USV at 0.33% | | | |
| 22 | BUBOND 60 at 356 ppm, | S. aureus 6538 | NZ/NI | 0 |
| | Emulsion A at 533 ppm, | | | |
| | R10800 at 0.67%, | | | |
| | and USV at 0.33% | | | |
| 23 | MICROBAN B | K. pneumoniae | NZ/I | 0 |
| | (Microban Products | | | |
| | Company) at 800 ppm, | | | |
| | R10800 at 0.67%, and | | | |
| | USV at 0.33% | | | |
| 24 | Microban B at 800 ppm, | S. aureus 6538 | NZ/NI | 0 |
| | R10800 at 0.67%, and | | | |
| | USV at 0.33% | | | |
| 25 | R10800 at 0.67%, and | K. pneumoniae | NZ/NI | 0 |
| | USV at 0.33% | | | |
| 26 | R10800 at 0.67%, and | S. aureus 6538 | NZ/NI | 0 |
| | USV at 0.33% | | | |
| 27 | PHMB at 237 ppm, FPS | K. pneumoniae | NZ/NI | 0 |
| | at 356 ppm, R10800 at | | | |
| | 0.67%, and USV at | | | |
| | 0.33% | | | |
| 28 | PHMB at 237 ppm, FPS | S. aureus 6538 | NZ/I | 0 |
| | at 356 ppm, R10800 at | | | |
| | 0.67%, and USV at | | | |
| | 0.33% | | | |
| 29 | PHMB at 237 ppm, | K. pneumoniae | NZ/NI | 0 |
| | Emulsion A at 356 ppm, | | | |
| | R10800 at 0.67%, and | | | |
| | USV at 0.33% | | | |
| 30 | PHMB at 237 ppm, | S. aureus 6538 | NZ/NI | 0 |
| | Emulsion A at 356 ppm, | | | |
| | R10800 at 0.67%, and | | | |
| | USV at 0.33% | | | |

Note that the percentages of R10800 and USV represent target loading levels on the cotton samples.

### Example 4

The efficacy of Emulsion A in cotton sample material was tested, alone and in combination with other agents. The AATCC Test Method 147, which is a known test method, was followed. The sample size of the cotton was 25 mm×75 mm rectangle. The growth medium was Nutrient Agar. Prior to treatment, the cotton was steam sterilized at 121° C. The samples were incubated at 37° C.±2° C. for 18 to 24 hours. The samples were each laundered 50 times unless indicated to the contrary. NZ means that no zone of inhibition surrounded the sample. NI means there was no inhibition of growth under the sample (if observable). The zone of inhibition was reported in millimeters.

| | | | | Size of |
|---|---|---|---|---|
| Sample | | | Zone | Zone |
| No. | Sample Description | Organism | Results | (mm) |
| 1 | Emulsion A at 1770 ppm, | K. pneumoniae | NZ/NI | 0 |
| | R10800 at 0.67% and | | | |
| | USV at 0.33% | | | |
| 2 | Emulsion A at 1770 ppm, | S. aureus 6538 | NZ/NI | 0 |
| | R10800 at 0.67% and | | | |
| | USV at 0.33% | | | |
| 3 | Emulsion A at 1180 ppm, | K. pneumoniae | NZ/NI | 0 |
| | R10800 at 0.67% and | | | |
| | USV at 0.33% | | | |
| 4 | Emulsion A at 1180 ppm, | S. aureus 6538 | I | 6 |
| | R10800 at 0.67% and | | | |
| | USV at 0.33% | | | |
| 5 | Emulsion A at 590 ppm, | K. pneumoniae | NZ/NI | 0 |
| | R10800 at 0.67% and | | | |
| | USV at 0.33% | | | |
| 6 | Emulsion A at 590 ppm, | S. aureus 6538 | I | 6 |
| | R10800 at 0.67% and | | | |
| | USV at 0.33% | | | |
| 7 | Emulsion A at 1770 ppm, | K. pneumoniae | NZ/NI | 0 |
| | R10800 at 0.92% and | | | |
| | USV at 0.08% | | | |
| 8 | Emulsion A at 1770 ppm, | S. aureus 6538 | NZ/NI | 0 |
| | R10800 at 0.92% and | | | |
| | USV at 0.08% | | | |
| 9 | Emulsion A at 1180 ppm, | K. pneumoniae | NZ/NI | 0 |
| | R10800 at 0.92% and | | | |
| | USV at 0.08% | | | |
| 10 | Emulsion A at 1180 ppm, | S. aureus 6538 | I | 3 |
| | R10800 at 0.92% and | | | |
| | USV at 0.08% | | | |
| 11 | Emulsion A at 590 ppm, | K. pneumoniae | NZ/NI | 0 |
| | R10800 at 0.92% and | | | |
| | USV at 0.08% | | | |
| 12 | Emulsion A at 590 ppm, | S. aureus 6538 | I | 4 |
| | R10800 at 0.92% and | | | |
| | USV at 0.08% | | | |
| 13 | Emulsion A at 1770 ppm, | K. pneumoniae | NZ/NI | 0 |
| | R10800 at 1.0% | | | |
| 14 | Emulsion A at 1770 ppm, | S. aureus 6538 | NZ/NI | 0 |
| | R10800 at 1.0% | | | |
| 15 | Emulsion A at 1180 ppm, | K. pneumoniae | NZ/NI | 0 |
| | R10800 at 1.0% | | | |
| 16 | Emulsion A at 1180 ppm, | S. aureus 6538 | I | 1 |
| | R10800 at 1.0% | | | |
| 17 | Emulsion A at 590 ppm, | K. pneumoniae | NZ/NI | 0 |
| | R10800 at 1.0% | | | |
| 18 | Emulsion A at 590 ppm, | S. aureus 6538 | NZ/NI | 0 |
| | R10800 at 1.0% | | | |
| 19 | Control | K. pneumoniae | NZ/NI | 0 |
| 20 | Control | S. aureus 6538 | NZ/NI | 0 |
| 21 | Control - no launderings | K. pneumoniae | NZ/NI | 0 |
| 22 | Control - no launderings | S. aureus 6538 | NZ/NI | 0 |

It will therefore be readily understood by those persons skilled in the art that the present invention is susceptible of broad utility and application. Many embodiments and adaptations of the present invention other than those herein described, as well as many variations, modifications and equivalent arrangements, will be apparent from or reasonably suggested by the present invention and the foregoing description thereof, without departing from the substance or scope of the present invention. Accordingly, while the present invention has been described herein in detail in relation to its preferred embodiment, it is to be understood that this disclosure is only illustrative and exemplary of the present invention and is made merely for purposes of providing a full and enabling disclosure of the invention. The foregoing disclosure is not intended or to be construed to limit the present invention or otherwise to exclude any such other embodiments, adaptations, variations, modifications and equivalent arrangements.

1. An antimicrobial composition comprising an emulsion, wherein said emulsion comprises:
a quaternary ammonium antimicrobial agent,
an alkyl phenol,
a styrenated phenol; and
water.

2. The antimicrobial composition according to claim 1, wherein
said quaternary ammonium antimicrobial agent is from about 15 wt. % to about 25 wt. % of the total composition,
said alkyl phenol is from about 20 wt. % to about 50 wt. % of the total composition, and
said styrenated phenol is from about 1 wt. % to about 10 wt. % of the total composition.

3. The antimicrobial composition according to claim 2, wherein said quaternary ammonium antimicrobial agent comprises an alkyl ammonium compound.

4. The antimicrobial composition according to claim 3, wherein said quaternary ammonium antimicrobial agent comprises an N-alkyl dimethylbenzyl ammonium compound.

5. The antimicrobial composition according to claim 1, further comprising an antifoaming agent.

6. The antimicrobial composition according to claim 2, wherein said alkyl phenol comprises at least one alkyl phenol having an alkyl group selected from the group consisting of C₇ alkyls, C₈ alkyls, C₉ alkyls, C₁₀ alkyls, and C₁₁ alkyls.

7. The antimicrobial composition according to claim 6, wherein said alkyl phenol comprises an alkyl phenol having a C₉ alkyl group.

8. The antimicrobial composition according to claim 1, further comprising an antifreeze agent.

9. The antimicrobial composition according to claim 1, wherein the quaternary ammonium antimicrobial agent is water insoluble.

10. A paint comprising the antimicrobial composition of claim 1.

11. A coating composition comprising the antimicrobial composition of claim 1.

12. A paper treated with the antimicrobial composition of claim 1.

13. A method of making an antimicrobial composition comprising:
blending an alkyl phenol with a quaternary ammonium antimicrobial agent,
heating the blended alkyl phenol and the quaternary ammonium antimicrobial agent,
admixing a quantity of a styrenated phenol, and
admixing a quantity of water.

14. The method according to claim 13, wherein the antimicrobial agent comprises an alkyl ammonium compound.

15. The method according to claim 14, wherein the antimicrobial agent comprises an N-alkyl dimethylbenzyl ammonium compound.

16. The method according to claim 13, wherein
the blended quaternary ammonium antimicrobial agent is present from about 15 wt. % to about 25 wt. % of the total composition;
the blended alkyl phenol is present from about 20 wt. % to about 50 wt. % of the total composition; and
the admixed styrenated phenol is present from about 1 wt. % to about 10 wt. % of the total composition.

17. The method according to claim 16, further comprising the step of admixing an antifoaming agent.

18. The method according to claim 13, further comprising admixing an antifreeze agent.

19. The method according to claim 13, wherein the alkyl phenol comprises at least one alkyl phenol having an alkyl group selected from the group consisting of C₇ alkyls, C₈ alkyls, C₉ alkyls, C₁₀ alkyls, and C₁₁ alkyls.

20. The method according to claim 13, wherein the blending of the antimicrobial agent with the alkyl phenol occurs under application of heat.

21. The method according to claim 13, wherein admixing the styrenated phenol occurs under application of heat.

22. The method according to claim 13, wherein admixing water occurs in absence of added heat.

23. A wallboard having antimicrobial properties, said wallboard comprising:
paper,
a quaternary ammonium antimicrobial agent,
an alkyl phenol,
a styrenated phenol; and
water.

24. Insulation exhibiting antimicrobial properties, said insulation comprising:
an insulating material,
paper,
a quaternary ammonium antimicrobial agent,
an alkyl phenol,
a styrenated phenol; and
water.

25. An antimicrobial composition comprising an emulsion, wherein said emulsion comprises:
a water insoluble quaternary ammonium antimicrobial agent,
an alkyl phenol,
a styrenated phenol; and
water.

26. The antimicrobial composition according to claim 25, wherein
said water insoluble quaternary ammonium antimicrobial agent is from about 15 wt. % to about 25 wt. % of the total composition,
said alkyl phenol is from about 20 wt. % to about 50 wt. % of the total composition, and
said styrenated phenol is from about 1 wt. % to about 10 wt. % of the total composition.

27. The antimicrobial composition according to claim 26, wherein said water insoluble quaternary ammonium antimicrobial agent comprises a non-halogenated benzyl substituted quaternary ammonium compound.

28. The antimicrobial composition according to claim 27, wherein said water insoluble quaternary ammonium antimicrobial agent comprises an N-alkyl dimethylbenzyl ammonium saccharinate.

29. The antimicrobial composition according to claim 25, further comprising an antifoaming agent.

30. The antimicrobial composition according to claim 25, wherein said alkyl phenol comprises at least one alkyl phenol having an alkyl group selected from the group consisting of C₇ alkyls, C₈ alkyls, C₉ alkyls, C₁₀ alkyls, and C₁₁ alkyls.

31. The antimicrobial composition according to claim 30, wherein said alkyl phenol comprises an alkyl phenol having a C₉ alkyl group.

32. The antimicrobial composition according to claim 25, further comprising an antifreeze agent.

33. The antimicrobial composition according to claim 25, wherein the water insoluble quaternary ammonium antimicrobial agent has the structure: 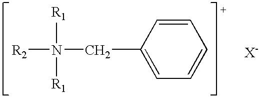
wherein
R₁ is methyl or hydroxyethyl;
R₂ is CₙH₂ₙ+1;
n is 8 to 18; and
X is an anionic species.

34. The antimicrobial composition according to claim 25, wherein the water insoluble quaternary ammonium antimicrobial agent is selected from the group consisting of N-alkyldimethyl benzyl ammonium saccharinate; alkyl dimethyl ethylbenzyl ammonium cyclohexylsulfamate; alkyl bis(2-hydroxyethyl)benzyl ammonium halide; alkyl dimethyl 1-napthylmethyl ammonium halide; alkyl dodecylbenzyl dimethyl ammonium halide; and alkylbenzyl trimethyl ammonium halide.

35. A paint comprising the antimicrobial composition of claim 25.

36. A coating composition comprising the antimicrobial composition of claim 25.

37. A paper treated with the antimicrobial composition of claim 25.

38. A method of making an antimicrobial composition comprising:
blending an alkyl phenol with a water insoluble quaternary ammonium antimicrobial agent,
heating the blended alkyl phenol and the quaternary ammonium antimicrobial agent,
admixing a quantity of a styrenated phenol, and
admixing a quantity of water.

39. The method according to claim 38, wherein the antimicrobial agent comprises a non-halogenated benzyl substituted ammonium compound.

40. The method according to claim 39, wherein the antimicrobial agent comprises a N-alkyl dimethylbenzyl ammonium saccharinate.

41. The method according to claim 38, wherein
the blended water insoluble quaternary ammonium antimicrobial agent is present from about 15 wt. % to about 25 wt. % of the total composition;
the blended alkyl phenol is present from about 20 wt. % to about 50 wt. % of the total composition; and
the admixed styrenated phenol is present from about 1 wt. % to about 10 wt. % of the total composition.

42. The method according to claim 41, further comprising the step of admixing an antifoaming agent.

43. The method according to claim 38, further comprising admixing an antifreeze agent.

44. The method according to claim 38, wherein the alkyl phenol comprises at least one alkyl phenol having an alkyl group selected from the group consisting of C₇ alkyls, C₈ alkyls, C₉ alkyls, C₁₀ alkyls, and C₁₁ alkyls.

45. The method according to claim 38, wherein the blending of the antimicrobial agent with the alkyl phenol occurs under application of heat.

46. The method according to claim 38, wherein admixing the styrenated phenol occurs under application of heat.

47. The method according to claim 38, wherein admixing water occurs in absence of added heat.

48. The method according to claim 38, wherein the water insoluble quaternary ammonium antimicrobial agent has the structure: 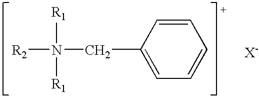
wherein
R₁ is methyl or hydroxyethyl;
R₂ is CₙH₂ₙ+1;
n is 8 to 18; and
X is an anionic species.

49. The method according to claim 38, wherein the water insoluble quaternary ammonium antimicrobial agent is selected from the group consisting of N-alkyldimethyl benzyl ammonium saccharinate; alkyl dimethyl ethylbenzyl ammonium cyclohexylsulfamate; alkyl bis(2-hydroxyethyl)benzyl ammonium halide; alkyl dimethyl 1-napthylmethyl ammonium halide; alkyl dodecylbenzyl dimethyl ammonium halide; and alkylbenzyl trimethyl ammonium halide.

50. A wallboard having antimicrobial properties, said wallboard comprising:
paper,
a water insoluble quaternary ammonium antimicrobial agent,
an alkyl phenol,
a styrenated phenol; and
water.

51. The wallboard according to claim 50, wherein the water insoluble quaternary ammonium antimicrobial agent has the structure: 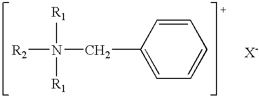
wherein
R₁ is methyl or hydroxyethyl;
R₂ is CₙH₂ₙ+1;
n is 8 to 18; and
X is an anionic species.

52. The wallboard according to claim 50, wherein the water insoluble quaternary ammonium antimicrobial agent is selected from the group consisting of N-alkyldimethyl benzyl ammonium saccharinate; alkyl dimethyl ethylbenzyl ammonium cyclohexylsulfamate; alkyl bis(2-hydroxyethyl)benzyl ammonium halide; alkyl dimethyl 1-napthylmethyl ammonium halide; alkyl dodecylbenzyl dimethyl ammonium halide; and alkylbenzyl trimethyl ammonium halide.

53. Insulation exhibiting antimicrobial properties, said insulation comprising:
an insulating material,
paper,
a water insoluble quaternary ammonium antimicrobial agent,
an alkyl phenol,
a styrenated phenol; and
water.

54. The insulation according to claim 53, wherein the water insoluble quaternary ammonium antimicrobial agent has the structure: 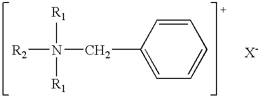
wherein
R₁ is methyl or hydroxyethyl;
R₂ is CₙH₂ₙ+1;
n is 8 to 18; and
X is an anionic species.

55. The insulation according to claim 53, wherein the water insoluble quaternary ammonium antimicrobial agent is selected from the group consisting of N-alkyldimethyl benzyl ammonium saccharinate; alkyl dimethyl ethylbenzyl ammonium cyclohexylsulfamate; alkyl bis(2-hydroxyethyl)benzyl ammonium halide; alkyl dimethyl 1-napthylmethyl ammonium halide; alkyl dodecylbenzyl dimethyl ammonium halide; and alkylbenzyl trimethyl ammonium halide.
